(19) United States
(12) Patent Application Publication
(10) Pub. No.: US 2025/0044869 A1
(43) Pub. Date: Feb. 6, 2025

(54) System for Dynamically Controlling an Output Environment

(71) Applicant: Ars Electronica Linz GmbH & Co KG, Linz (AT)

(72) Inventors: Susanne KIESENHOFER, Linz (AT); Anna OELSCH, Linz (AT); Daniel RAMMER, Linz (AT)

(21) Appl. No.: 18/787,900

(22) Filed: Jul. 29, 2024

(30) Foreign Application Priority Data
Jul. 31, 2023 (AT) A 50612/2023

(51) Int. Cl. G06F 3/01; G06F 3/0346

(52) U.S. Cl. CPC G06F 3/015; G06F 3/0346

(57) ABSTRACT

A system for dynamically controlling an output environment comprising a computer unit and at least one output means controllable by the computer unit, wherein the system is configured to output an output generated by the computer unit via the output means in the output environment, wherein the system comprises at least two wearables each attachable to one person, wherein the wearables are connected to the computer unit and configured to substantially continually detect and transmit to the computer unit at least one physiological parameter of the person wearing the respective wearable, and the computer unit is configured to control the output based on all physiological parameters detected by the wearables.

### FIELD OF THE INVENTION

The invention relates to a system for dynamically controlling an output environment, comprising a computer unit and at least one output means controllable by the computer unit, the system being configured for outputting an output generated by the computer unit via the output means in the output environment.

### BACKGROUND

There are various embodiment variants of systems for controlling an output environment available on the market. Systems known according to the prior art comprise a computer unit and at least one output means controllable by the computer unit. This output means may, for example, be implemented in the form of virtual or augmented reality glasses. In addition, there are known systems having large screens or projectors as output means, which are used in walk-in rooms for simulating an artificial environment. The output means is thus configured for outputting an output generated by the computer unit in the output environment and thereby generating a virtual environment. Such systems are known on the market under the keywords of virtual reality, augmented reality, mixed reality and/or extended reality systems.

Such systems can also be interactive, for example be controlled via user input. User input is usually made via a controller or gesture control, by means of which a user can generate commands for the computer unit.

One disadvantage of such systems is, however, that they only have limited feedback mechanisms between the user and the displayed content. Usually, the user has to make an active input in order to give commands to the computer unit. Furthermore, systems according to the prior art allow a very limited extend of user group involvement, which is usually only possible as entire groups and not as individuals.

The invention is based on the task of providing a system for dynamically controlling an output environment that avoids these disadvantages of the prior art.

According to the invention, this task is solved by a system comprising at least two wearables each attachable to one person, wherein the wearables are connected to the computer unit and configured to substantially continually detect and transmit to the computer unit at least one physiological parameter of the person wearing the respective wearable. In addition, the computer unit is configured to control the output based on all physiological parameters detected by the wearables.

This achieves the advantage that each person of a user group is integrated into the system according to the invention, and the at least one physiological parameter of each person is automatically and substantially continuously detected. Thus, the output environment is subconsciously and automatically controlled by all users together, which allows the generation of a continuous input-output loop. Substantially continuously within the framework of the invention means the detection of a physiological parameter with a statistical or dynamic sampling interval.

Preferably, the system according to the invention has detection means arranged within the area of the output environment and connected to the computer unit for detecting environmental conditions within the output environment and/or physiological parameters of persons located within the output environment, wherein the computer unit is configured to control the output via all physiological parameters detected by the wearables and the detection means and via the environmental conditions detected by the detection means. This achieves the advantage that environmental conditions in the output environment also have an effect on the output.

According to the preferred embodiment variant of the system according to the invention, the physiological parameter of the person detected by the respective wearable comprises the pulse, body temperature, electrodermal activity, oxygen saturation in the blood, EEG data, ECG data, audio data, acceleration measurement data, position data, magnetic data, location data, eye position, viewing direction, and/or breathing rate of the person. This achieves the advantage that a wide range of parameters of the user group, which, inter alia, allow conclusions to be drawn about unconscious and subconscious, biological and physiological processes, are provided for controlling the system according to the invention.

Preferably, the computer unit is additionally configured to create a statistical profile from all detected physiological parameters detected by the wearables of all persons wearing at least one wearable and to control the output based on the statistical profile. This allows that the system according to the invention is not only controlled based on the individual physiological parameters of the user group, but that group dynamic parameters of the user group can also be detected and used to control the system. Thus, the system can automatically respond to a reaction of the entire user group. Preferably, the creation of a statistical profile comprises the detection of a mean value, a weighted mean value, a skewness, and/or a standard deviation of the detected physiological parameters of the persons.

Advantageously, the wearables each comprise at least one output means connected to the computer unit, which is configured to output an output generated by the computer unit via the output means of the wearable directly to the person wearing the wearable. This allows transmitting direct feedback to a specific person. This output means of the wearables can comprise a display device, a loudspeaker and/or a haptic output device, such as a vibration unit. Furthermore, the display device can, for example, comprise a heating element or a ventilator.

According to the preferred embodiment variant of the system according to the invention, each wearable comprises a wristband, a chest strap, glasses, a headband, a hat, a headwear, a neckband and/or a necklace. This provides for a plurality of different attachment options for the wearable on the respective person.

Preferably, the system according to the invention additionally comprises location detection sensors connected to the computer unit, which are configured to detect a location of the persons in the output environment. Preferably, the computer unit is also configured to control the output based on the location of the at least one person. This achieves the advantage that the output means can be controlled by the computer unit based on the location of the persons in the output environment. Preferably, the location detection sensors comprise LiDAR sensors, cameras, network triangulation devices, Bluetooth triangulation devices and/or UWB triangulation devices. In addition, the wearables can also comprise inertial measurement units, which can be combined with one or more of the location detection sensors mentioned above. This achieves the advantage that a pose or posture or a motion sequence of a person can be linked to the absolute or relative location of the person in the room.

The wearables are preferably connected to the computer unit via WIFI, Bluetooth, a cable, a mobile network and/or direct radio connection. This provides for a plurality of connection options.

The wearables can additionally each comprise at least one input means for the manual input of a control command and for transmitting the control command to the computer unit. This provides the individual users with active input options.

### BRIEF DESCRIPTION OF THE DRAWING

Advantageous embodiments of the system according to the invention and alternative embodiment variants will now be explained in further detail with reference to the FIGURES.

FIG. 1 shows a schematic diagram of a preferred embodiment variant of a system according to the invention for dynamically controlling an output environment.

### DETAILED DESCRIPTION OF THE PREFERRED EMBODIMENTS

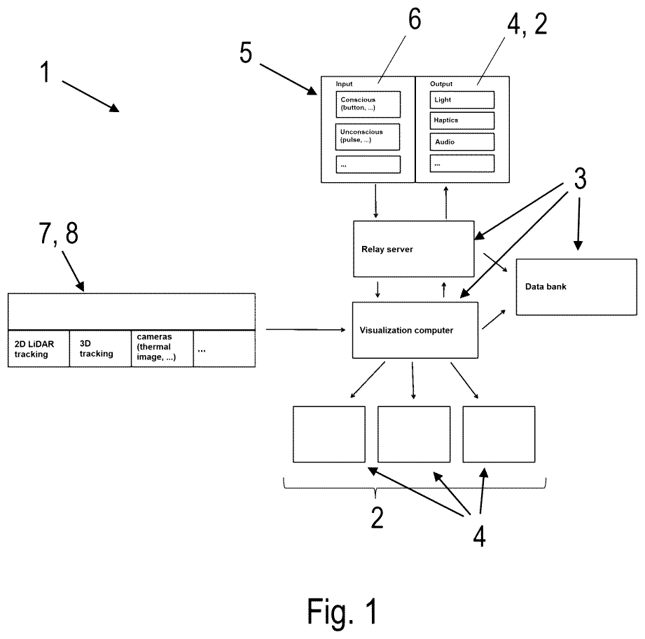

The system according to the invention 1 for dynamically controlling an output environment 2 shown as a preferred embodiment variant in FIG. 1 comprises a computer unit 3 and at least one output means 4 controllable by the computer unit. The computer unit 3 can, for example, be provided in the form of a server connected to the output means 4. The output means 4 can, for example, be a projection unit, a screen, or also virtual reality glasses or augmented reality glasses. Naturally, the system according to the invention 1 can, as shown in FIG. 1, also comprise several output means 4 in the output environment 2. According to a preferred embodiment of the invention, projection units, screens, and loudspeakers are used as output means 4. The system according to the invention 1 is configured to output an output generated by the computer unit 3 via the output means 4 in the output environment 2. Here, the output environment 2 can be a real room in which the output means 4 are installed. Alternatively, the output environment 2 can also be completely or partly virtual and be represented by one or more output means 4, such as virtual reality glasses or one or more screens or projectors. Augmented reality, mixed reality and or extended reality glasses are also usable as output means 4 within the framework of the invention. According to one embodiment variant, the output environment 2 can thus be a room in which the output means 4 are installed or into which the output means 4 can be brought be the users. Thus, a plurality of different output means 4 can be used to shape the experience of a user of the system according to the invention 1. According to the invention, the system 1 comprises at least two wearables 5 attachable to a person, the wearables 5 being connected to the computer unit 3 and being configured to substantially continuously detect at least one physiological parameter of a person wearing the respective wearable 5 and transmit it to the computer unit 3. The output means 4 can also be provided on the wearables 5. The computer unit 3 is preferably provided in the form of a server. The computer unit 3 is configured to control the output based on all physiological parameters detected by the wearables 5. Here, the detected parameters can, among other things, comprise pulse, body temperature, electrodermal activity, oxygen saturation in the blood, EEG data, ECG data, audio data, acceleration measurement data, position data, location data, magnetic data, eye position, viewing direction, and/or breathing rate. For this, the wearables 5 have corresponding sensors, such as inertial measurement units and/or biosensors detecting these parameters. Furthermore, the wearable 5 can, for example, be provided in the form of glasses and comprise an eye movement sensor, which is not visible in FIG. 1. It is thus possible to determine how much attention which person pays to the output generated in the output environment 2. With the detected parameters it is, for example, possible to also determine an emotional reaction of the person wearing the respective wearable 5. According to the invention, substantially continuous detection refers to detection of the mentioned parameters with a conventional sampling interval of such sensors generally known to a person skilled in the art.

This achieves the advantage that the output, and thus the content represented in the output environment 2, can be controlled based on data of each individual person using the system according to the invention 1. This allows the creation of a feedback loop between the output in the output environment 2 and the reaction of the users, which allows responding either to reactions of the entire user group or to reactions of only one member of the user group.

The wearables 5 can, for example, comprise a wristband, a chest strap, glasses, a headband, a headwear, a hat, a neckband and/or a necklace. In addition, it is also possible that a wearable 5 comprises more than one of these components. Preferably, the wearables 5 are connected to the computer unit 3 via WIFI, Bluetooth, a cable, a mobile network and/or direct radio connection. Further alternative connection methods are generally known to a person skilled in the art. The wearables 5 can also each comprise at least one input means 6 for manually inputting a control command and transmitting the control command to the computer unit 3. This input means 6 can, for example, be provided in the form of one or more touch screens or one or more push buttons. The input means 6 can also be provided in the form of one or more microphones with a software for voice recognition and voice control. This allows the persons to make active inputs to the system. According to one embodiment variant of the system according to the invention 1, handheld devices such as game controllers that are connected to the computer unit 3 can be provided in addition to the wearables 5, which handheld devices can alternatively or in addition to the wearables 5 also have such input means 6.

Preferably, the computer unit 3 is configured to create a statistical profile of all persons wearing at least one wearable 5 from all physiological parameters detected by the wearables 5 and to control the output based on the statistical profile. Any mathematical methodology from statistics can be used for creating the statistical profile in order to generate data of interest. This allows the detection of group dynamic parameters of the user group and their use for controlling the system according to the invention 1. Preferably, the creation of a statistical profile comprises the detection of a mean value, a weighted mean value, a skewness, and/or a standard deviation of the detected physiological parameters of the persons. Thus, the system according to the invention 1 is also suitable for the use in, for example, sociological studies or for analyzing group dynamic processes.

The wearables 5 themselves can also each comprise at least one output means 4 connected to the computer unit 3. I can be used for outputting an output generated by the computer unit 3 via the output means 4 of the wearables 5 directly to the person wearing the wearable 5. Preferably, this output means 4 of the wearables 5 comprises a display device, a loudspeaker and/or a haptic output device, such as a vibration unit. In addition, the display device can, for example, comprise a heating element or a ventilator. Thus, outputs can be directly output to a single person of a group of users of the system according to the invention 1.

For a further improvement of the output of the system according to the invention 1, the system 1 can preferably comprise location detection sensors 7. These location detection sensors 7 detect a location of the persons in the output environment 2. For this purpose, the location detection sensors 7 can, for example, comprise LiDAR sensors or cameras, such as RGB, greyscale and/or depth cameras. 3D point cloud tracking means are also implementable as location detection sensors 7 within the framework of the system according to the invention 1. The location detection sensors 7 are also connected to the computer unit 3, and the computer unit 3 preferably controls the content by means of the locations of all persons in the output environment 2 detected by the location detection sensors 7. Alternatively, marker-based tracking can also be used, where the persons are provided with active or passive markers that are captured by cameras. Thus, the exact viewing direction of every person can be detected in combination with 3D glasses as wearables 5 and an eye-tracking system. The locations of the persons can, for example, be detected by means of image analysis of the images from the cameras. This allows, for example, also the detection of the postures of individual or all persons. Alternative location detection sensors 7 are generally known to a person skilled in the art. By detecting the location of every person in the output environment 2, the output can be adapted to this location by the computer unit 3.

Preferably, the system according to the invention 1 also has detection means 8 arranged in the output environment 2 and connected to the computer unit 3 in order to detect environmental conditions in the output environment 2 and/or physiological parameters of the persons in the output environment 2. These detection means 8 can, for example, be formed by thermal imaging cameras, thermometers, hygrometers, etc. Here, the computer unit 3 is configured to control the output based on all physiological parameters detected by the wearables 5 and the detection means 8 and on the environmental conditions detected by the detection means 8. This achieves the advantage that the environmental conditions in the output environment 2 also have an effect on the output. In addition, this allows, for example, influencing the locations and parameters of individual persons in a group by means of a feedback loop or to deducing a relationship between the persons within the group based on the locations and physiological parameters of the individual persons. Furthermore, the system according to the invention 1 can be configured to store the detected parameters of the persons. This can, for example, be implemented by means of a data bank that is part of the computer unit 3 or connected to the computer unit 3. These are thus available for a later analysis. According to one embodiment variant of the system according to the invention 1, the detected parameters are transmitted from the computer unit 3 to the wearables 5 of the persons and displayed to them.

Consequently, the persons can view the parameters and thus, for example, react to specific states of one or more other persons within the user group. Due to the various feedback options of the system according to the invention 1, it is, for example, suitable as a mediation tool or also as a training tool for treating states of anxiety. Furthermore, the system according to the invention 1 can strengthen the feeling of solidarity within a user group. The audibility and visualization of processes in the body of each person that are usually invisible can be used for strengthening and training the social competences, emotional intelligence and attentiveness of every person. In addition, the system 1 provides support in overcoming language and cultural barriers between persons using the system 1.

According to one embodiment variant of the system according to the invention 1, the computer unit 3 can, as shown in FIG. 1, comprise one or more computers, wherein one of the computers serves as a relay server and one of the computers is configured as a visualization computer. The computer unit 3 can also be implemented as a server on which several virtual machines are defined, which are each configured as a relay server and/or visualization computer. According to this embodiment variant of the system according to the invention 1, the wearables 5 detect the parameters and calculate average values from these. The raw and average values are then transmitted to the relay server. The relay server collects all data of all users from the wearables 5. The relay server stores all these data and provides them to the visualization computer. The relay server may transmit these data also to other interfaces. The relay server or the visualization computer also stores all data in a data bank. The visualization computer connects to the relay server and receives all previously collected data and then calculates useful data from these for controlling the output means 4, for example values for the playback of sounds and images, such as volume, pitch and/or speed.

Within the framework of one embodiment variant of the system according to the invention 1, users can be assigned at several levels. For example, a wearable 5 that is worn by a user or person, can illuminate in a specific color, and the parts of the output, e.g. the projection, representing the data of this person are shown or coded in the same color. This is referred to as representation in the output environment 2. This coding can also be achieved by other means, such as FIGURES, words, shapes, and images. In order to allow the system according to the invention 1 to connect a wearable 5 and the respective data to other parts of the system 1, such as the 2D LiDAR tracking, e.g. for assigning it, the users go to a marked location in the output environment 2 or in a room. The marking of this location corresponds to a marking on the wearable 5 worn by the respective person, for example by means of the color.

If a user enters one of the color-coded and numbered circles, they are assigned a tracking ID, for example by the 2D LiDAR tracking system. This allows the assignment of the respective wearable 5 to the locations in the room. In addition, the users thus know which representation in the output environment 2 is assigned to them. This can, for example, be achieved by projections of circles with different colors in the output environment 2, which may additionally be numbered. The colors and/or numbers of the respective wearables 5 then corresponds to the colors and/or numbers of the circles.

The output or projection can also be designed in a way that users have no possibility of identifying with certain graphic or auditive elements. For example, only an average value of all users might be made visible or audible.

Alternatively, the localization may be provided directly on the wearable, with the means described herein, so that separate assignments are not necessary.

What is claimed is:

1. A system for dynamically controlling an output environment comprising:
a computer unit and an output means controllable by the computer unit, wherein the system is configured to output an output generated by the computer unit via the output means in the output environment,
wherein:
the system comprises at least two wearables each attachable to one person, wherein the at least two wearables are connected to the computer unit and configured to substantially continually detect and transmit to the computer unit at least one physiological parameter of a person wearing a respective wearable, and the computer unit is configured to control the output based on all physiological parameters detected by the at least two wearables.

2. The system according to claim 1, wherein the system comprises detection means arranged within an area of the output environment and connected to the computer unit for detecting environmental conditions within the output environment and/or physiological parameters of persons located within the output environment, wherein the computer unit is configured to control the output via all physiological parameters detected by the at least two wearables and the detection means and via the environmental conditions detected by the detection means.

3. The system according to claim 1, wherein the physiological parameter of the person detected by the respective wearable comprises a pulse, body temperature, electrodermal activity, oxygen saturation in a blood, EEG data, ECG data, audio data, acceleration measurement data, position data, location data, magnetic data, eye position, viewing direction, and/or breathing rate of the person.

4. The system according to claim 1, wherein the computer unit is configured to create a statistical profile from all detected physiological parameters of all persons wearing at least one wearable and to control the output based on the statistical profile.

5. The system according to claim 1, wherein the at least two wearables each comprise at least one output means connected to the computer unit, which is configured to output an output generated by the computer unit via the output means of the at least two wearables directly to the person wearing the respective wearable.

6. The system according to claim 5, wherein the output means of the at least two wearables comprises a display device, a loudspeaker, a heating element, a ventilator and/or a haptic output device, such as a vibration unit.

7. The system according to claim 1, wherein each wearable comprises a wristband, a chest strap, glasses, a headband, a headwear, a hat, a neckband and/or a necklace.

8. The system according to claim 1, wherein the system comprises location detection sensors connected to the computer unit, which are configured to detect a location of at least one person in the output environment, and the computer unit is configured to control the output based on the location of the at least one person.

9. The system according to claim 8, wherein the location detection sensors comprise LiDAR sensors, cameras, network triangulation devices, Bluetooth triangulation devices, and/or UWB triangulation devices.

10. The system according to claim 1, wherein the at least two wearables are connected to the computer unit via WIFI, Bluetooth, a cable, a mobile network and/or direct radio connection.

11. The system according to claim 1, wherein the at least two wearables each comprise an input means for a manual input of a control command and for transmitting the control command to the computer unit.

12. The system according to claim 4, wherein a creation of the statistical profile comprises a detection of a mean value, a weighted mean value, a skewness and/or a standard deviation of the detected physiological parameters of the person.

13. The system according to claim 2, wherein the physiological parameter of the person detected by the respective wearable comprises a pulse, body temperature, electrodermal activity, oxygen saturation in a blood, EEG data, ECG data, audio data, acceleration measurement data, position data, location data, magnetic data, eye position, viewing direction, and/or breathing rate of the person.
